(19) United States
(12) Patent Application Publication
(10) Pub. No.: US 2025/0381063 A1
(43) Pub. Date: Dec. 18, 2025

(54) Integrated Patient Warming And Positioning System With Filtered Exhaust and Controlled Airflow

(71) Applicant: Infinitus Medical Technologies LLC, Holly Springs, NC (US)

(72) Inventors: David J. Gomez, Holly Springs, NC (US); Nathan Winder, San Jose, CA (US)

(21) Appl. No.: 19/240,071

(22) Filed: Jun. 17, 2025

Related U.S. Application Data
(60) Provisional application No. 63/660,945, filed on Jun. 17, 2024.

(51) Int. Cl. A61F 7/00; A61G 13/12

(52) U.S. Cl. CPC A61F 7/0085; A61F 7/0053; A61G 13/1265; A61F 2007/0055; A61F 2007/0086; A61F 2007/0091; A61F 2007/0096

(57) ABSTRACT

A filtered exhaust heat pad integrates warming, positioning, and handling via a bladder with one or more lumens, a universal inlet, and a filtered exhaust outlet. The system delivers conductive warming (37-42° C.), adjustable rigidity, and turbulence reduction, supporting pressure offloading (≤32 mmHg) and clean-to-clean transfers. The system is configurable for orthopedic, abdominal, chest, pelvic, head, and neck surgeries, and the system integrates with blankets, minimizing contamination and enhancing interoperability.

### TECHNICAL FIELD

The invention pertains to medical devices for surgical care, specifically an integrated system combining patient warming, positioning, and safe handling. The present invention provides a self-contained bladder with internal airflow lumens, a universal air inlet, and a filtered exhaust port with flow control. The invention delivers conductive warming, minimizes airborne contamination, and ensures compatibility with surgical positioning systems.

### BACKGROUND ART

Surgical procedures require precise patient positioning, effective warming, and safe handling to ensure safety and optimal outcomes. Current standalone, proprietary systems lack interoperability, causing inefficiencies and compromised care. Positioning devices (e.g., grounding pads, RF pads, gel pads) obstruct conductive warming systems, reducing thermal efficacy and hindering tissue offloading for pressure management, particularly in friction-based platforms for gravity-dependent positions (e.g., Trendelenburg, lateral tilt). Reduced friction risks patient instability, pressure ulcers, and nerve damage.

Forced air warming systems, used for normothermia, generate turbulent airflow (e.g., 0.5-2 m/s), potentially increasing infection risks by dispersing airborne pathogens, especially in orthopedic, abdominal, pelvic, head, and neck surgeries. Delaying warming until after surgical prepping and draping mitigates airflow issues but delays normothermia (core temperature≥36° C.), risking complications like increased morbidity, prolonged recovery, infections, and impaired wound healing. For instance, even a 1° C. drop while waiting for surgical prep, draping, or incision, may push a patient toward mild hypothermia (below 36° C.), especially if sustained. This condition can impair the ability of the body to regulate temperature, particularly under anesthesia, which suppresses thermoregulation. And due to “catch up” phase of warming vs. surgical operative time, a 1° C. drop may trigger shivering, increasing oxygen consumption and metabolic demand, which can stress the cardiovascular system in the post operative care phase. Furthermore, the standard of care for shivering patients is not just continued warming, but requires opioid type drugs such as Demerol as the “K” opioid receptor is known for its anti-shivering impact, thus delaying discharge and increasing costs.

What is needed is an integrated system addressing infection risks, interoperability, pressure management, and thermal efficiency.

### BRIEF SUMMARY OF THE INVENTION

With parenthetical reference to the corresponding parts, portions or surfaces of the disclosed embodiment, merely for the purposes of illustration and not by way of limitation, the present invention meets the above described need by providing a device that integrates patient warming, positioning, pressure management, and safe handling. The device comprises a biocompatible bladder with one or more internal airflow lumens, a multi-layered structural foam core for conductive warming, a universal air inlet compatible with forced air warming units, and a filtered exhaust port with bacterial/viral or HEPA/ULPA filtration (≥99.97% for ≥0.3 μm). A precision flow control valve regulates pressure (0.05-0.3 kPa) and exhaust flow (0-50 L/min) to enable adjustment of the rigidity of the device. The system integrates with high-friction positioning devices, supports pressure offloading (≤32 mmHg), and facilitates air-assisted transfers. The system is configurable for orthopedic, abdominal, chest, pelvic, head, and neck surgeries. The technology may also be used to improve typical surgical blanket designs placed on top of the patient by using the technology to maximize tissue contact and warming efficiency.

The present invention mitigates forced outflow by providing a low flow air delivery system within a sealed blanket made of fabric, plastic, or non-woven material. The design reduces energy consumption and enables conductive warming rather than relying solely on convection air warming for the patient. The filtered aperture for outflow can be adjusted to control the balance between air outflow and inflation of the conductive device, making it compatible with standard forced air blowers with higher air delivery capacities.

The system provides a range of patient air warming blankets that can be positioned either on top or underneath the patient. Each blanket may be equipped with filtered air outflow and an optional accessory cap that can be connected to wall suction. This configuration facilitates conductive warmth through the turbulent flow of warm air within an airtight fabric, non-woven, or plastic enclosure, while if connected to suction, eliminates aberrant airflow within the perioperative environment.

The warming pad for placement under the patient features an adjustable outflow aperture that can increase resistance to reduce air outflow. This adjustment allows for the management of modestly inflated pressure to support the tissue of the patient. Additionally, the under body pad may include an internal support system made of soft foam or similar material which is soft yet structurally supportive to prevent collapse, enhance air circulation and improve conductive heating. The outflow aperture can also be linked to room air suction to minimize air turbulence in the operating room.

In another aspect, the enclosed, forced air and conductive warming system includes a HEPA filtered outflow flanged flexible tubing connected to a standard anesthesia, CPAP, or respiratory filtration accessory. This accessory filtration device may be custom fitted to a pigtail or flexible mounting tube, with one or two ports extending from within the enclosed conductive warming apparatus. This configuration enables the adjustment of rotational resistance in air flow which is significant for balancing inflation and managing the filtered air outflow either into the ambient environment or through wall suction.

### BRIEF DESCRIPTION OF THE DRAWINGS

FIG. 1 is a schematic elevation view of one embodiment of the system of the present invention.

FIG. 2 is a top plan view of an alternate embodiment of the present invention.

FIG. 3 is a top plan view of another alternate embodiment of the present invention.

FIG. 4 is a perspective view of a filtered exhaust heat pad according to the present invention.

FIG. 5 is another perspective view of the filtered exhaust heat pad shown in FIG. 4.

### DESCRIPTION OF THE PREFERRED EMBODIMENTS

At the outset, it should be clearly understood that like reference numerals are intended to identify the same structural elements, portions or surfaces consistently throughout the several drawing figures, as such elements, portions or surfaces may be further described or explained by the entire written specification, of which this detailed description is an integral part. Unless otherwise indicated, the drawings are intended to be read (e.g., cross-hatching, arrangement of parts, proportion, debris, etc.) together with the specification, and are to be considered a portion of the entire written description of this invention. As used in the following description, the terms “horizontal”, “vertical”, “left”, “right”, “up” and “down”, as well as adjectival and adverbial derivatives thereof, (e.g., “horizontally”, “rightwardly”, “upwardly”, etc.), simply refer to the orientation of the illustrated structure as the particular drawing figure faces the reader. Similarly, the terms “inwardly” and “outwardly” generally refer to the orientation of a surface relative to its axis of elongation, or of rotation, as appropriate.

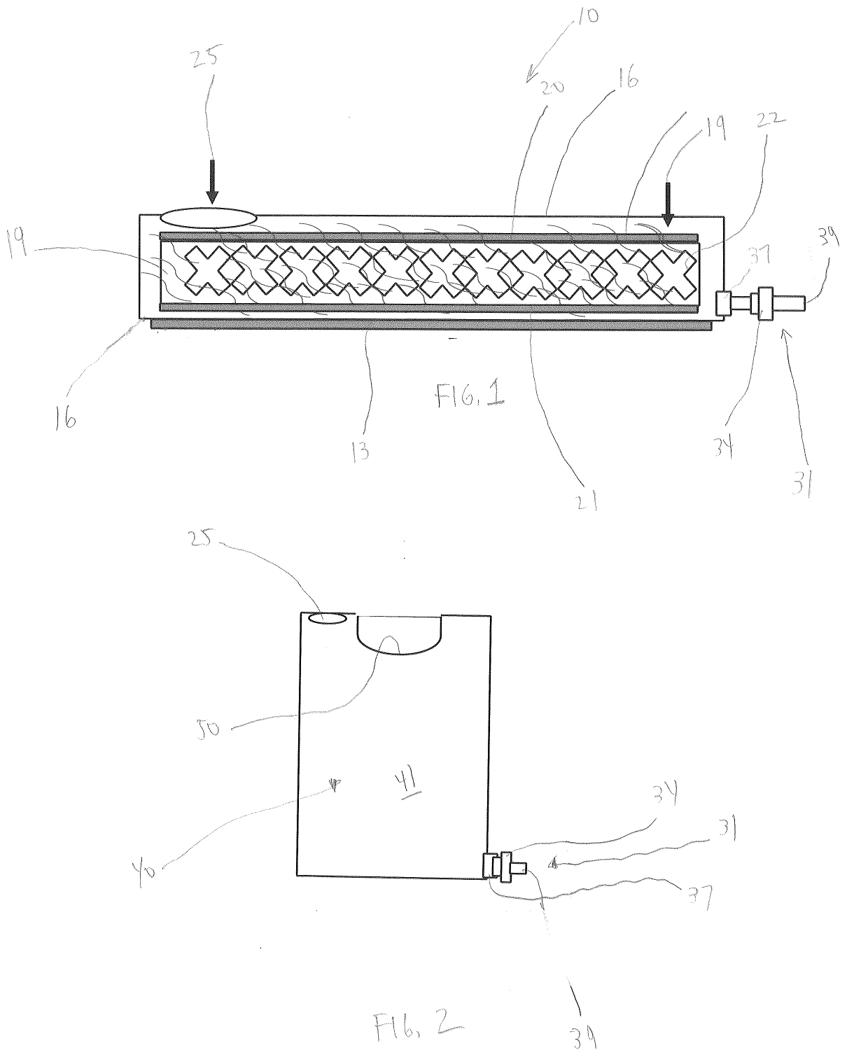

Referring now to the drawings, and more particularly to FIG. 1 thereof, this invention provides a flexible warming blanket 10. In one embodiment, the blanket 10 may be configured as follows: 80-150 cm long, 50-100 cm wide, and 2-5 cm thick. The blanket 10 may incorporate a viscoelastic or standard polyurethane foam layer 13 between the patient and the outer surface 16 of the blanket 10. The foam layer 13 may have a density 40-80 kg/m³, pore size 0.5-2 mm, and thermal conductivity 0.05-0.1 W/m·K. The foam layer 13 enhances conductive heat absorption (heat flux≥100 W/m²), ensuring uniform contact (≥90%) and pressure offloading (≤32 mmHg). The outer layer of the blanket 10 may be provided with a polyurethane-coated nylon (burst strength≥500 kPa) for durability and infection control.

A single or multi-layered foam core 19 enhances structural integrity, pressure offloading (≤32 mmHg), and airflow turbulence reduction (Reynolds number≤2000). The foam core 19, which may comprise foam layers 20 and 21, maintains a low center of gravity (≤5 cm from table surface) for stability. Adjustable apertures (e.g., 0.2-3 mm variable pores, baffle spacing 0.5-2 cm) optimize inflation dynamics, pressure distribution, and thermal conductivity (≥0.1 W/m·K). Foam options, used singly or in laminated combinations, include: (a) polyurethane foam with a density of 40-80 kg/m³, pore size 0.5-2 mm, thermal conductivity 0.05-0.1 W/m·K, and indentation force deflection (IFD) 5-50 N, for contouring and turbulence dampening (vibration reduction≤5 Hz). (b) open-cell polyether foam: density 20-50 kg/m³, pore size 0.2-1.5 mm, porosity≥90%, compressive strength 10-30 kPa, air permeability 50-150 L/m²·s, for airflow control and stability under 250 kg loads. (c) reticulated polyester foam: density 25-60 kg/m³, pore size 1-3 mm, 95-98% open cells, thermal conductivity≥0.08 W/m·K, for low airflow resistance (pressure drop≤0.02 kPa) and thermal distribution. (d) silicone-based foam: density 30-70 kg/m³, pore size 0.3-1.8 mm, thermal stability (37-43° C.), compressive set≤5%, conductivity 0.06-0.12 W/m·K, for sterilization durability (autoclave at 121° C.). (e) EVA foam: density 30-100 kg/m³, pore size 0.1-1 mm, resilience≥95%, conductivity 0.03-0.08 W/m·K (enhanced with graphene additives), for structural rigidity. (f) polyethylene foam: density 20-50 kg/m³, pore size 0.2-1.2 mm, compressive strength 15-40 kPa, water absorption<1%, conductivity 0.04-0.09 W/m·K, for sterilization compatibility.

The present invention may also utilize foam combinations. Laminated layers (e.g., viscoelastic or standard non VE polyurethane base with reticulated polyester top) optimize pressure offloading, thermal transfer, and airflow. Adjustable apertures may be provided via variable-density molding or laser-cut channels (0.1-0.5 mm precision).

One or more lumens 22 or a network of interconnected channels, which may be 0.5-2 cm wide and 0.5-5 L total volume, are formed by heat-sealed or RF-welded baffles (0.1-0.2 mm thick) made from bladder-compatible materials. As shown in FIG. 1, configurations (parallel, serpentine, grid) ensure uniform air distribution (velocity variation≤10%) and heat transfer (37-42° C., thermal efficiency≥80%). The one or more lumens or channels 22 may minimize pressure drop (≤0.05 kPa) and maintain laminar flow (Reynolds number<2000) to reduce turbulence.

An inlet port 25 which may have a 2-5 cm diameter may be provided with a bayonet or threaded locking connector (not shown, torque resistance≥5 Nm). The connector may be compatible with forced air warming units (e.g., with a flow of 20-50 L/min, pressure 0.1 -0.5 kPa). The connector may be constructed from polycarbonate (impact strength≥50 KJ/m²), ABS plastic, or reinforced cardboard (burst strength≥500 kPa), it could include a check valve to prevent backflow.

An outlet port 31 which may be 1-3 cm diameter may be provided with a bacterial/viral or HEPA/ULPA filter 34 (≥99.97% for ≥0.3 μm, ≥99.999% for ≥0.12 μm, per EN 1822), matching CPAP/BiPAP standards, or anesthesia filter standards. The outlet port 31 may be pivotable (0-180°, friction torque 0.5-1 Nm) to direct airflow or connectable to suction devices/house vacuum via ISO 5356-1 compliant fittings (leak rate<0.01 L/min). An adjustable aperture (0.5-3 cm, manual dial) eliminates aberrant airflow (turbulence intensity ≤5%). Acoustic dampening reduces noise (≤10 dB at 1 m).

A rotary or slide valve 37 (e.g., stopcock, resolution 0.1 L/min) regulates exhaust flow (0-50 L/min) and internal pressure (0.05-0.3 kPa). Optional piezoelectric sensors (accuracy ±0.01 kPa) provide real-time pressure/flow feedback, enabling manual or automated control via a microcontroller (e.g., 8-bit, 16 MHz).

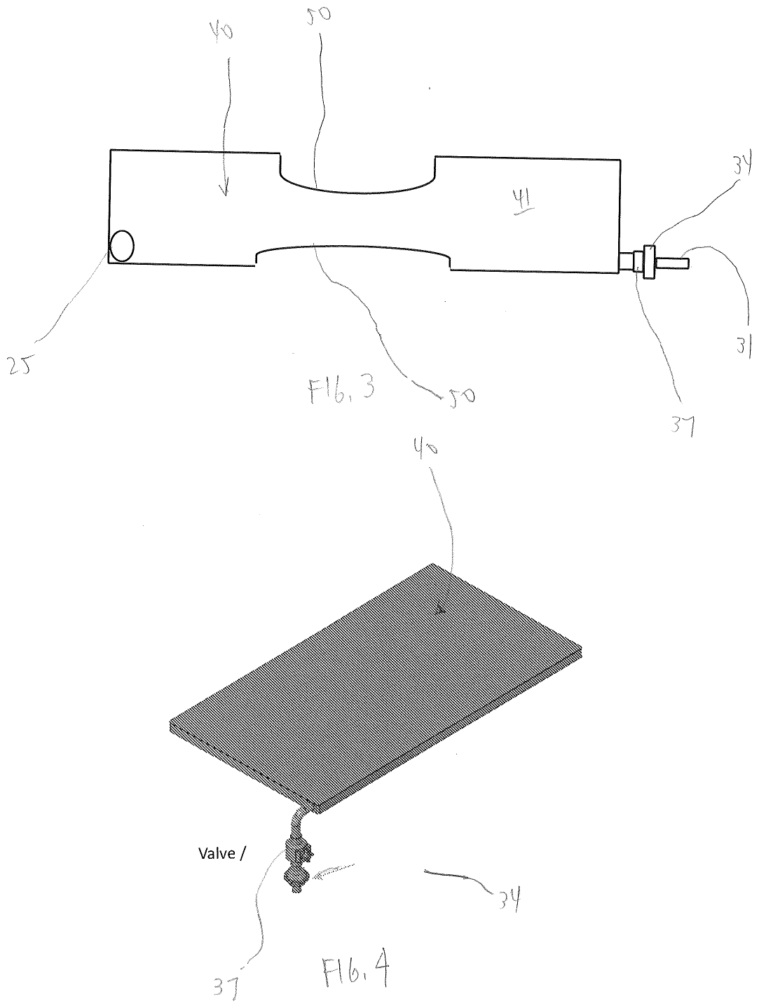

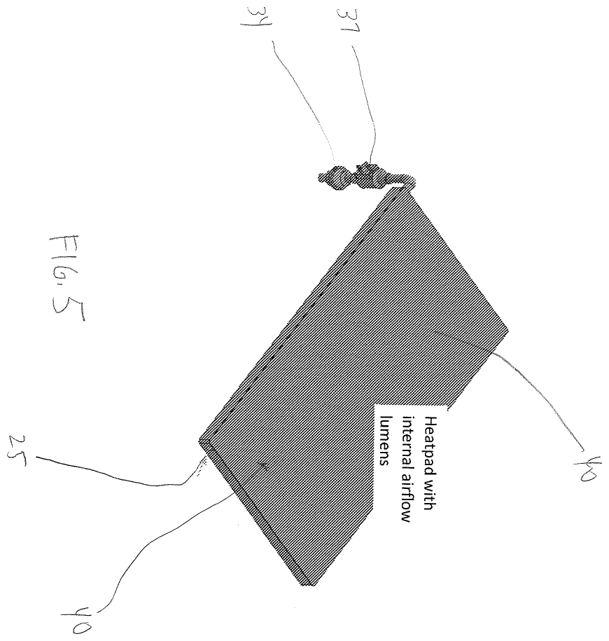

Turning to FIGS. 2-5, a flexible or semi-rigid underbody bladder 40 which in one embodiment may be 50-200 cm long, 30-100 cm wide, and 5-15 cm thick may be constructed from biocompatible polyurethane, nylon, or silicone composites (tensile strength 20-30 MPa). Sealed or permeable with micro-perforations (0.1-0.5 mm, 10-100/cm²), it includes a foam core 19 for pressure offloading (≤32 mmHg), turbulence reduction (Reynolds number<2000), and stability (center of gravity≤5 cm). The exterior surface 41 of the bladder 40 may be coated with silicone or polyurethane (friction coefficient≥0.6, ASTM D1894) for traction (frictional force≥50 N) when integrated with high-friction positioning devices (e.g., foam pads, surgical mattresses). The surface supports pressure management (compressive strength 10-50 kPa, deformation≤5% at 250 kg) and thermal conductivity (≥0.1 W/m·K). Optional antimicrobial coatings (e.g., silver-ion, efficacy≥99.9% per ISO 22196) may be added to enhance infection control.

In operation, a forced air warming unit delivers air (35-43° C., 20-50 L/min, 0.1-0.5 kPa) through the inlet 25 into the lumen 22.

Heat Distribution: Lumen 22 and foam core 19 distribute air, heating the bladder surface 41 (37-42° C., uniformity±1° C.) for conductive warming (heat flux≥100 W/m²). The foam core ensures consistent patient contact (contact area≥80%) and thermal efficiency (≥80%).

Exhaust Control: The filtered exhaust port 31 removes ≥99.97% of particles≥0.3 μm. The valve 37 adjusts flow to maintain pressure, optimizing rigidity (Young's modulus 10-100 kPa). Suction connection 39 (e.g., 10-20 kPa vacuum) may a closed system, eliminating open-air discharge (air velocity<0.1 m/s at 1 m).

Positioning Integration: The bladder 40, placed under or between a positioning device (not shown) and patient, maintains traction (≥50 N) for stability in gravity-dependent positions. Its low-profile design (5-15 cm) and foam core 19 reduce tissue pressure (≤32 mmHg, per ISO 14708), ensuring a low center of gravity for stability.

Transfer Compatibility: The bladder 40 secures to air-assisted transfer devices via hook-and-loop (shear strength≥20 N/cm²), pressure-sensitive adhesives (peel strength≥5 N/cm), or similar detachable utility. It detaches for clean-to-clean transfers, adhering to ISO 17664 sterilization protocols.

The technical specifications are as follows: Operating Temperature: 37-42° C. (±0.5° C., adjustable via warming unit); Airflow Capacity: 20-50 L/min, pressure 0.05-0.3 kPa (burst pressure≥1 kPa); Filtration Efficiency: ≥99.97% for ≥0.3 μm, ≥99.999% for ≥0.12 μm, acoustic dampening≤10 dB (A-weighted, 1 m); Durability: Supports 250 kg (safety factor 2), withstands autoclaving (121° C., 15 psi, 30 min) or ethylene oxide sterilization; Dimensions: Configurable (e.g., 100×50×3 cm adult; 50×30×2 cm pediatric/veterinary); Weight: 0.5-2 kg (material-dependent); Power Requirements: None (external warming unit).

The system may be integrated with surgical systems such as positioning platforms: compatible with high-friction pads/substrates (e.g., SMS, taffeta, friction coefficient≥0.6) per U.S. patents application Ser. No. 17/083,725, Ser. No. 16/964,567 which are incorporated herein by reference. The positioning platforms ensure fixation in Trendelenburg (tilt≤30°) or lateral tilt) (≤20°).

Air-Assisted Transfer Devices: Attachable via detachable interfaces (e.g., hook-and-loop, PSA, etc.) for patient movement (transfer force≤200 N) and clean-to-clean transfers (ISO 17664 compliance).

Forced Air Warming Units: Universal inlet 25 supports standard units (e.g., standard commercial forced air warmer blowers, flow 20-50 L/min) or proprietary low-flow systems (10-20 L/min) via adapters (leak rate<0.01 L/min).

Surgical Blankets 10: Configurable for under/over patient use, integral with blankets 10 using identical bladder 40, lumens 22, and filtered exhaust technology (filter 34 with filtration ≥99.97%) to maximize tissue contact (≥90% surface area), while utilizing an outer foam 13 interface to improve heat conduction and pressure management on the patient's skin surface.

Surgical Applications: Suitable for orthopedic (e.g., hip replacement), abdominal (e.g., laparotomy), chest (e.g., thoracotomy), pelvic, head, and neck surgeries, with size/rigidity adjustments (e.g., inflatable for pediatric, rigid for spinal procedures).

The system provides the follow advantages.

Infection Control: Filtered exhaust with adjustable suction eliminates turbulent airflow (velocity<0.1 m/s) and pathogens, potentially reducing infection risks (e.g., SSI rates≤1%).

Interoperability: Universal inlet 25 and modular design ensure compatibility with existing equipment (e.g., ISO 80369 compliance).

Pressure Management: Foam core 19 and surface 16 reduce tissue pressure (≤32 mmHg), preventing pressure ulcers and nerve damage, additive to overlaying positioners.

Versatility: Configurable for diverse surgeries, with blanket 10 integration for enhanced warming (heat transfer≥100 W/m²).

Patient Safety: Maintains normothermia (≤36° C., per AORN guidelines) and secure positioning, reducing morbidity (e.g., cardiac output drop≤5%).

Ease of Use: Lightweight, sterilizable, adaptable for pediatric/veterinary use, with color-coded components for identification.

The following features may also be added to the system.

Sensor Integration: Embedded temperature (thermocouple, ±0.1° C.), pressure (piezoelectric, ±0.01 kPa), and airflow sensors (hot-wire, ±0.1 L/min), with Bluetooth/Wi-Fi connectivity (IEEE 802.15.1) for surgical monitor integration.

Modular Configurations: Detachable lumen sections (snap-fit connectors, release force ≤10 N) or foam core inserts (magnetic alignment) for customization.

Antimicrobial Coatings: Silver-ion or quaternary ammonium coatings (efficacy ≥99.9%, ISO 22196) on bladder 40/foam 13.

Color-Coding: ISO 26825-compliant colors for size/application (e.g., blue for adult, green for pediatric).

Smart Control: Microcontroller-based valve adjustment (PID algorithm, response time ≤1 s) for automated pressure/flow optimization.

The present system has many key differences in comparison to typical forced air warming systems that have been used.

The mechanism for heat transfer differs between the present system and traditional forced air warming systems.

Typical forced air warming systems deliver warm air (35-43° C.) through a perforated blanket, relying on convective heat transfer to warm the patient's skin surface. These systems offer broad coverage but generate turbulent airflow (0.5-2 m/s), increasing infection risks due to airborne pathogen dispersion.

In contrast, the filter exhaust heat pad of the present invention uses forced warm air to heat the surface of bladder 40 (37-42° C.), which conductively transfers heat to the patient or contacting devices. It minimizes turbulent airflow but may have limited contact area, potentially reducing overall heat transfer compared to convective systems.

Forced air warming systems risk surgical site infections (SSIs) due to turbulent airflow mobilizing pathogens (e.g., Staphylococcus aureus). Studies indicate SSI rates may increase by 1-3% in orthopedic surgeries with forced air warming.

Conductive systems, like the heat pad of the present invention, use filtered exhaust (≥99.97% for ≥0.3 μm) and optional suction to eliminate open-air discharge, significantly reducing contamination risks.

Forced air warming blankets cover large areas (e.g., torso, limbs), delivering 100-200 W/m² of heat flux. However, efficiency drops in areas with poor blanket contact.

Conductive systems rely on direct contact (contact area≥80%), with heat flux≥100 W/m² but potentially lower total heat delivery if contact is limited to the bladder's surface (e.g., 0.5-1 m² vs. 1-2 m² for blankets).

Forced air warming blankets may interfere with surgical access or positioning devices, especially in orthopedic or pelvic surgeries.

Conductive systems integrate with high-friction positioning devices (friction≥50 N), supporting pressure offloading (≤32 mmHg) and stability in gravity-dependent positions (e.g., Trendelenburg).

The conductive warming system of the present invention matches the thermal performance and coverage of forced air warming while maintaining its advantages in infection control and positioning compatibility. The following strategies optimize the design of the filtered exhaust heat pad.

The bladder 40 is designed to conform to the patient's anatomy using a viscoelastic or pneumatic foam core 19 (e.g., polyurethane, IFD 20-50 N) that adapts to body contours, maximizing contact area (≥90%). The adjustable apertures on the heat pad (0.2-3 mm) ensure uniform inflation, enhancing contact without compromising rigidity.

The bladder 40 is paired with a surgical blanket 10 using identical technology (e.g., lumens 22, filtered exhaust 31) placed over the patient, as described in the patent. This dual-layer approach (under and over patient) mimics the broad coverage of forced air blankets, achieving a combined contact area of 1-1.5 m².

The thermal conductivity of the present invention may be enhanced as follows.

The system may incorporate thermally conductive additives (e.g., graphene, carbon nanotubes) into the surface of the bladder 40 or foam core 19, increasing thermal conductivity from 0.1 W/m·K to 0.2-0.5 W/m·K. This boosts heat flux to ≥150 W/m², approaching forced air warming levels (100-200 W/m²).

The present invention may also use a multi-layered foam core 19 (e.g., viscoelastic polyurethane base with reticulated polyester top) to balance thermal transfer and airflow, ensuring uniform surface temperature (37-42° C., +0.5° C.).

The present invention may integrate embedded thermocouples (accuracy ±0.1° C.) and a microcontroller (e.g., 8-bit, 16 MHz) to adjust inlet air temperature (35-43° C.) based on real-time patient skin temperature feedback, maintaining normothermia (≥36° C.). This compensates for the slower response for conductive warming compared to convective systems.

The exhaust port 31 of the present invention, with a HEPA/ULPA filter 34 (≥99.97% for ≥0.3 μm), removes pathogens, unlike the open discharge associated with traditional forced air warming. Enhanced filtration with multi-stage media (e.g., pre-filter+HEPA) allows the system to handle high flow rates (20-50 L/min) without clogging (pressure drop≤0.02 kPa).

An adjustable aperture suction option (0.5-3 cm) may be used to eliminate aberrant airflow (turbulence intensity≤5%), achieving operating room air velocity<0.1 m/s, comparable to laminar flow systems (ISO 14644-1 Class 5).

The exhaust port 31 of the present invention may be connected to house vacuum or suction devices (10-20 kPa) via ISO 5356-1 fittings, creating a fully closed system. This eliminates open-air discharge, matching the infection control of conductive systems like electric blankets while retaining the dynamic heating of forced air systems.

The present invention may provide for lumens 22 with serpentine or grid patterns to maintain laminar flow (Reynolds number<2000), reducing internal turbulence. The open-cell structure of the foam core 19 (porosity≥90%) further dampens airflow variations (velocity uniformity ±10%).

The present invention may provide a modular blanket 10 system with identical bladder 40 technology (lumen 22, foam core 19, filtered exhaust 31) for placement over the torso, limbs, or head of the patient. The blanket 10 connects to the same warming unit via a Y-splitter (leak rate<0.01 L/min), delivering 20-30 L/min to each component (underbody bladder 40+blanket 10).

The system of the present invention may also use flexible silicone-based foam (Shore A 30-50) in the blanket for drapability, ensuring ≥90% tissue contact. This mimics forced air warming's coverage (1-2 m²) while maintaining conductive heat transfer (≥100 W/m²).

:

The system of the present invention may provide bladder 40 sizes for specific applications (e.g., 100×50 cm for adult torso, 50×30 cm for pediatric, 150×80 cm for bariatric). Adjustable rigidity (Young's modulus 10-100 kPa) via flow control (0.05-0.3 kPa) supports diverse surgeries (e.g., rigid for spinal, inflatable for pelvic).

The present invention may also incorporate cutouts 50 or detachable sections (snap-fit, release force≤10 N) in the bladder 40/blanket 10 for surgical access (e.g., hip traction in orthopedics, abdominal ports in laparoscopy), thereby matching adaptability of forced air warming systems.

The system of the present invention maintains the high-friction surface of the heat pad (coefficient≥0.6, frictional force≥50 N) to integrate with positioning devices (e.g., foam pads, per U.S. patent application Ser. No. 17/083,725, Ser. No. 16/964,567). This ensures stability in Trendelenburg (tilt)≤30° or lateral tilt) (≤20°, unlike forced air blankets, which may slip (friction<0.4).

The low center of gravity of the foam core 19 (≤5 cm) and pressure offloading (≤32 mmHg, along with the friction or stiction coefficient of the foam) prevent patient sliding, matching or exceeding forced air warming's positioning compatibility.

The system of the present invention uses a multi-layered foam core 19 (e.g., EVA or reticulated foam for rigidity, polyurethane for contouring) to achieve compressive strength (10-50 kPa) and tissue pressure reduction (≤32 mmHg), additive to overlaying positioners. This prevents pressure ulcers and nerve damage, a limitation in forced air systems with minimal offloading.

Forced air warming delivers 100-200 W of total heat, depending on blanket size (1-2 m²). To match this, the bladder 40 and optional blanket 10 of the system of the present invention provides ≥150 W/m² across a combined area of 1-1.5 m², achieving 150-225 W total heat. This result is feasible with enhanced thermal conductivity (0.2-0.5 W/m·K) and optimized airflow (30-50 L/min).

Forced air warming reaches target skin temperature (36-38° C.) in 10-15 minutes. Conductive warming may take 15-20 minutes due to slower heat diffusion. The time difference may be reduced by pre-warming the bladder 40 (37-42° C.) before patient placement and using dynamic temperature control (response time≤1 s) to adjust heat output.

Forced air warming units consume 500-1000 W. The system of the present invention, using the same units, maintains efficiency (≥80%) by minimizing heat loss through sealed lumens 22 and filtered exhaust 31. Closed-loop suction further reduces energy waste by recycling air pressure.

Forced air warming increases airborne particle counts by 10-100% in operating rooms (ISO 14644-1 Class 7). The filtration of the filtered exhaust heat pad (≥99.97%) and suction reduce particle counts to Class 5 levels (<3520 particles/m³ for ≥0.5 μm), matching or exceeding laminar flow environments.

The system of the present invention may be provided with silver-ion or quaternary ammonium coatings (efficacy≥99.9%, ISO 22196) for the bladder 40 and blanket 10 surfaces, reducing microbial colonization risk, a feature absent in most forced air blankets.

The Heatpad's lightweight design (0.5-2 kg) and color-coded components (ISO 26825) match forced air warming's simplicity. Training requirements are minimal, as it uses existing warming units.

The bladder 40 of the present invention may withstand autoclaving (121° C., 15 psi, 30 min) or ethylene oxide, like forced air blankets. Disposable options (gamma-irradiated, 25-40 kGy) ensure cost-effectiveness for single-use scenarios.

To balance performance, in one embodiment the system of the present invention may be configured as follows:

Bladder: 100×50 cm underbody bladder 40 with a laminated foam core 19 (viscoelastic polyurethane base, reticulated polyester top, density 40-60 kg/m³, conductivity 0.2 W/m·K).

Blanket: 120×80 cm overbody blanket 10 with silicone-based foam (Shore A 30, contact area≥90%), connected via Y-splitter.

Airflow: 40 L/min total (20 L/min bladder 40, 20 L/min blanket 10), pressure 0.1-0.2 kPa, delivered by a 3M™ Bair Hugger™ unit.

Exhaust: Dual filtered ports 31 (HEPA, ≥99.97%) with suction (15 kPa vacuum), maintaining air velocity<0.1 m/s.

Control: Microcontroller with thermocouples adjusts temperature (37-42° C.) and valve flow (10-40 L/min) for normothermia (≥36° C.) and rigidity (Young's modulus 50 kPa).

Positioning: Integrated with high-friction foam pad (friction≥50 N), supporting Trendelenburg) (30° with pressure offloading (≤32 mmHg).

This setup delivers 150-200 W total heat, matches forced air warming's coverage (1.3 m²), and eliminates turbulence, achieving thermal and infection control equivalence.

Balancing a forced air conductive warming system like the system of the present invention with typical forced air warming is achieved by optimizing heat transfer (contact area, conductivity), minimizing turbulence (filtration, suction), expanding coverage (blanket integration), and ensuring positioning compatibility (friction, offloading). The design of the system, with its foam core 19, filtered exhaust 31, and universal inlet 25, can match the thermal performance of traditional forced air warming products (150-200 W, normothermia≥36° C.) while surpassing its infection control (particle reduction to Class 5) and positioning capabilities (stability in 30° tilt). By implementing modular blankets 10, dynamic controls, and rigorous validation, the system achieves equivalence with enhanced safety and versatility for surgical applications.

The present invention contemplates that many changes and modifications may be made. Therefore, while the presently-preferred form of the integrated patient warming and positioning system with filtered exhaust and controlled airflow has been shown and described, and several modifications and alternatives discussed, persons skilled in this art will readily appreciate that various additional changes and modifications may be made without departing from the spirit of the invention, as defined and differentiated by the following claims.

1. A patient warming and positioning system for use with a patient supported on a surgical table, the system comprising:
a bladder having a first lumen defined therein configured for conductive warming, the bladder having a foam core disposed therein, the foam core configured for pressure offloading, the bladder disposed underneath the patient;
a first universal air inlet disposed in fluid communication with the first lumen in the bladder, the first universal air inlet configured for conveying a warming fluid into the first lumen;
a first filtered exhaust port with adjustable suction disposed in fluid communication with the first lumen in the bladder;
a warming blanket having a second lumen disposed in fluid communication with a second universal air inlet and a second filtered exhaust port, the warming blanket disposed above the patient.

2. The system of claim 1, further comprising a foam layer disposed between the patient and the warming blanket, the foam layer configured to enhance conductive heat transfer.

3. The system of claim 1, wherein the one of the first and second filtered exhaust port is pivotable between zero and one hundred eighty degrees.

4. The system of claim 1, wherein one of the first and second filtered exhaust ports is configured for connection to a vacuum suction-connectable exhaust.

5. The system of claim 1, wherein the foam core is comprised of reticulated polyurethane, polyether, polyester, silicone, EVA, or polyethylene, singly or combined.

6. The system of claim 1, wherein the system is configured for one of orthopedic, abdominal, chest, pelvic, head, and neck surgeries.

7. The system of claim 1, wherein the bladder removably attaches to an air assisted transfer device and is configured to support pressure management and clean-to-clean transfers.

8. The system of claim 1, wherein the exterior of the bladder may be coated with silicone or polyurethane for traction when the system is integrated with a high-friction positioning device.

9. The system of claim 1, wherein the bladder or foam core is provided with an antimicrobial coating to enhance infection control.

10. The system of claim 1, further comprising one of embedded temperature, pressure, and airflow sensors, configured for surgical monitor integration.

11. The system of claim 1, further comprising a microcontroller-based valve adjustment for automated pressure/flow optimization.

12. The system of claim 1, wherein the bladder is shaped to conform to the anatomy of the patient using a foam core that adapts to body contours of the patient to maximize contact area.

13. The system of claim 1, further comprising thermally conductive additives disposed in the surface of the bladder or foam core.

14. The system of claim 1, further comprising embedded thermocouples and a microcontroller to adjust inlet air temperature based on real-time patient skin temperature feedback.

15. The system of claim 1, wherein the foam core comprises a viscoelastic polyurethane base with a reticulated polyester top.

16. The system of claim 1, wherein the bladder or blanket comprises cutouts or detachable sections for surgical access.

17. A patient warming and positioning system for use on a patient supported on a surgical table, the system comprising:
a bladder having a first lumen defined therein, the first lumen configured for conductive warming and having a foam core configured for pressure offloading;
a first universal air inlet disposed in fluid communication with the first lumen in the bladder for conveying warming fluid into the first lumen;
a first filtered exhaust port with adjustable suction disposed in fluid communication with the first lumen in the bladder;
a high-friction surface disposed beneath the bladder, the high friction surface configured to engage with a patient positioning device on the surgical table;
an upper warming blanket having a second lumen disposed in fluid communication with a second universal air inlet and a second filtered exhaust port, the foam layer configured to enhance conductive heat transfer by improving thermal absorbance and ensuring patient contact, optimizing warming efficiency while supporting pressure offloading; and, a viscoelastic polyurethane foam layer disposed between the patient and the upper warming blanket.

18. The system of claim 17, further comprising a microcontroller-based valve adjustment for automated pressure/flow optimization.

19. The system of claim 17, further comprising embedded thermocouples and a microcontroller to adjust inlet air temperature based on real-time patient skin temperature feedback.

20. A method for warming and positioning a patient on a surgical table, the method comprising:
providing a bladder with a lumen, an inlet, and a filtered exhaust outlet;
delivering a warm fluid to the inlet of the bladder;
positioning the bladder under or over a surgical positioning device;
adjusting the exhaust/suction at the filtered exhaust outlet to minimize turbulence;
warming the patient with conductive heat from the bladder; and, adjusting the pressure inside the bladder to support pressure offloading.
